(19) United States
(12) Patent Application Publication
(10) Pub. No.: US 2006/0064162 A1
(43) Pub. Date: Mar. 23, 2006

(54) 333Intraocular lens device

(76) Inventor: William L. Klima, Fredericksburg, VA (US)

Correspondence Address: KLIMA LAW OFFICES, P.L.L.C., P.O. Box 2855, Stafford, VA, 22555-2855, US

(21) Appl. No.: 10/943,405

(22) Filed: Sep. 17, 2004

(51) Int. Cl. A61F 2/16

(52) U.S. Cl. 623/6.37; 623/6.44; 623/6.46; 623/6.56

(57) ABSTRACT

A intraocular lens device including a lens portion connected to a haptic portion by a leg portion, preferably a bent leg portion. The intraocular lens device is preferably an accommodating intraocular lens device configured to move a lens portion located in a first plane towards the haptic portion located in a second plane located a predetermined distance from the first plane when tension forces are applied to opposite ends of the haptic portion.

### FIELD OF THE INVENTION

The present invention is directed to an intraocular lens device, preferably an accommodating intraocular lens device.

### BACKGROUND OF THE INVENTION

There exists many types, arrangements, designs or otherwise configurations of intraocular lens devices, and more recently accommodating intraocular lens devices.

Intraocular lens devices have been very successful for use in cataract surgery after the natural crystalline lens has been removed from the capsular bag located in the posterior chamber of the eye. More recently, intraocular lens are being configured for refractive correction of the eye such as the implantable contact lens (icl) or phakic refractive lens (prl) configured to be implanted between the natural crystalline lens and the iris, or the Artisan claw lens configured for implantation in the anterior chamber of the eye.

There exists a need for an intraocular lens device configured to be adjustable in vivo, after implantation in the eye, for example, to change or adjust the fit, refractive power, size, shape, aspherical characteristics, configuration the lens portion, configuration of the haptic portion, or change or adjust other aspects or characteristics of the intraocular lens device. Further, there is a need for an accommodating intraocular lens device configured to provide enhanced accommodation, or for providing an accommodation multiplier or amplifier.

### SUMMARY OF THE INVENTION

An object of the present invention is to provide an improved intraocular lens device.

A second object of the present invention is to provide an improved accommodating intraocular lens device.

A third object of the present invention is to provide an adjustable intraocular device configured to be adjusted in vivo, after implantation in the eye.

A fourth object of the present invention is to provide a pre-stressed intraocular lens device.

A fifth object of the present invention is to provide an improved accommodating intraocular lens device configured to accommodate by application of tension force applied on the haptic portion or portions, in particular on edges of the plate haptic portion or portions of the accommodating intraocular lens device.

The present invention is directed to an intraocular lens device, preferably to an accommodating intraocular lens device. The intraocular lens device according to the present invention is preferably an adjustable, pre-stressed and/or tension actuated intraocular lens device.

A preferred embodiment of the intraocular lens device according to the present invention is configured to be changed or adjustable in vivo, after implantation into the eye. Specifically an intraocular lens device according to the present invention can be a lens only, a lens portion in combination with looped type haptic portion(s), lens portion in combination with a plate type haptic portion or portions, and a lens portion in combination with both a plate type haptic portion and a loop type haptic portion.

The adjustable intraocular lens device according to the present invention can be configured to adjust one or more parts, components, points, areas, portions, or the entire intraocular lens device itself in vivo, after implantation in the eye. For example, the fit of the lens portion, the fit of the haptic portion, the fit of both the lens portion and the haptic portion, the size of the lens portion, the size of the haptic portion, the size of both the lens portion and haptic portion, the shape of the lens portion, the shape of the haptic portion, the shape of both the lens portion and haptic portion, the surface properties (e.g. surface tension, surface energy, surface finish, molecular or atomic changes to the surface and/or interior portions, color of surface and/or interior portions, light transmittance of surface and/or interior portions, light reflectance of surface, adherence properties of the surface with surrounding tissue, lubrication properties of surface, hydrophobic or hydrophilic properties of surface, cross-linking of surface, structural stiffness of lens portion, structural stiffness of haptic portion, structural stiffness of both lens portion and haptic portion, change in length, width, and/or thickness of lens portion, change of length, width and/or thickness of haptic portion, change of length, width and/or thickness of both lens portion and haptic portion, color of lens portion, color of haptic portion, color of both lens portion and haptic portion, change of hardness of lens portion, change of hardness of haptic portion, change of hardness of both lens portion and haptic portion, change of symmetry of lens portion, change of symmetry of haptic portion, change of symmetry of both lens portion and haptic portion, aspheric correction of lens portion, a toric correction of lens portion can be adjusted in vivo, after implantation of the intraocular lens device in the eye.

The intraocular lens device according to the present invention can be changed one time, many times, changed at different times, changed over a period of time (e.g. aging), changed periodically or on a schedule, changed on demand, or changed in other modes. Preferably the intraocular lens device according to the present invention is changed or adjusted by means of electromagnetic radiation applied to one or more portions of the intraocular lens device. For example, electromagnetic radiation (e.g. heat infrared, ultraviolet, laser, x-rays) can be applied into the eye from a source located outside of the eye. Alternatively, the intraocular lens device according to the present invention can be provided by an energy source (e.g. battery) implanted within the eye and/or body of the patient. Alternatively, or in addition, electrical components can be provided within the intraocular lens device, eye and/or body to change, convert, control, regulate or otherwise interface with an energy source outside of the eye and provide a source of energy within the eye and/or body (e.g. energy transfer by spinning magnets, electromagentic capacitance, telepathy, radio wave transmission through tissue, or other know methods).

The intraocular lens device according to the present invention can be configured from a single material used to make both the lens portion and haptic portion, or can be a composite using different materials. For example, the lens portion is mainly made from a non-deformable material (e.g. PMMA), or more preferably from a soft, pliable, deformable or otherwise resilient material (e.g. silicon polymer, acrylic polymer, collagen containing polymer, polyurethane) and the haptic portion(s) is or are made from a deformable or resilient material, preferably having a higher tensile strength (e.g. polyester, polypropylene, polyamide, polysulfone, polymethyl methacrylate) verses the lens material.

The lens portion and/or haptic portion can be configured to be adjustable by use of electromagnetic radiation in a variety of ways. For example, the lens portion can be adjusted by treating a specific point(s), area(s), volume(s), surface(s), axis(es), plane(s) with electromagnetic radiation causing additional polymerization or cross-linking of polymer(s), degradation of polymer or breaking cross-linking of polymer, annealing, welding, heating, softening, hardening, loss of tensile strength, increase of tensile strength, decrease in hardness, increase in hardness, formation of bubbles, elimination of bubbles, formation of voids, elimination of voids, tension forces, compression forces, torsional forces, increase opacification, decrease opacification, increase light transmittance, decrease light transmittance, increase curvature, decrease curvature, and changes or adjustments to many other characteristics or features of the lens portion and/or haptic portion(s).

The lens portion can be provided with one or more lens inserts or lens implants made of different material versus the lens portion itself to provide points, surfaces, planes and/or volumes of interfaces therebetween. When electromagnetic radiation is focused at the lens implant or lens insert, the lens implant or lens insert can be heated, cooled, stressed, chemically altered and/or physically altered to change the characteristics or features of the lens portion. Alternatively, application of electromatic radiation at the interface at points, surfaces, axes, planes and/or volumes can provide changes or adjustments to the lens portion due to nature of the interface(s). The lens implants or lens inserts can be macroscopic or microscopic, for example, on the order of millimeters to micons in size, or smaller. For example, the lens implants or lens inserts can be made of solids, gels, powder, particles, microcapsules, cells, strands, strings, threads, rods, filiments, and other types of known small objects arranged in a particular arrangement (e.g. matrix arrangement, circular, radial) within the lens portion. In addition, additives such as metal atoms, metal containing molecules, organic molecules and/or inorganic molecules can be added to the polymer used to make the lens portion, which can further catalyze cross-linking of polymer, faciliate heating or cooling of points, areas or volumes within the molecular matrix of the lens portion.

The lens implants or inserts and the haptic implants or inserts can be made of silicone polymer, polymethyl methacrylate (PMMA), polyimide, polyiimide, polyester, polypropylene, polycarbonate, fiberglass, Kevlar, graphite, carbon, boron, composite, collagen containing polymer, or other suitable know material.

In some embodiments, for example, an interior portion or portions of the lens portion are softened or possibly even liquefied by application of electromagnetic radiation focused at these interior portions (e.g. to model the natural crystalline lens). On a very sophisticated basis, layers of different hardness from the center of the lens portion can be formed by application of laser light by three-dimensional mapping and focusing of the laser light at specific points, areas, planes within the interior portion of the lens portion. U.S. Pat. No. ______ and U.S. Pat. No. ______ are incorporated herein by reference regarding the use of electromagnetic radiation for adjusting a lens in vivo, after implantation in the eye.

Another preferred intraocular lens device according to the present invention is a pre-stressed intraocular lens device. Specifically, the intraocular lens is configured, arrangement, made and/or otherwise manufactured in a manner so that the intraocular lens or portions thereof are in a pre-stressed condition after formation thereof. For example, a silicon deformable intraocular lens is made in a manner so as to provide stress (e.g. tension, compression and/or torsion) in the mold and during the curing process resulting in a finished intraocular lens having internal and/or surface stresses in a resting condition. Alternatively, a deformable intraocular lens is made from a dehydrated blank, and the blank is pre-stressed during machining of the lens surfaces prior to hydration. As an additional type of pre-stressing, the intraocular lens device is provided with one or more implants or inserts that are pre-stressed (e.g. tension, compression and/or torsion) while the intraocular lens device is being molded, cured, formed or otherwise being made. For example, an insert is placed under tension in a mold while the intraocular lens device is molded around the insert. After molding, the tension is relieved on the insert placing portions of the intraocular lens device under compression. Alternatively, an insert is placed under compression while molding the intraocular lens portion, and again the compression force is relieved after formation thereof creating tension forces within the lens portion.

A further embodiment of the intraocular lens according to the present invention is a intraocular lens device, in particular, an accommodating lens device, in which the configuration or conformation of the intraocular lens is changed in vivo, after implantation in the eye, by application of pulling or tension forces applied to the edges of the intraocular lens device by the eye. For example, the intraocular lens device is configured with the lens portion located in a different plane relative to the haptic portion, and when tension forces are applied to the edges of the intraocular lens, the lens portion moves towards or into the same plane as the haptic portion(s).

The above three preferred embodiments of the intraocular lens device according to the present invention, including an adjustable intraocular lens device, a pre-stressed intraocular lens device, and an intraocular lens device capable of changing configuration by application of tension force can be separate features or aspects of the intraocular lens device according to the present invention, or can be utilized in various combinations.

The lens insert(s) can be made of optically clear material, and/or can be sized sufficiently smaller at to not significantly interfere with the refractive properties of the lens portion of the intraocular lens.

### BRIEF DESCRIPTION OF THE DRAWINGS

FIG. 1 is a perspective view of a preferred embodiment of the intraocular lens device according to the present invention.

FIG. 2 is a side elevational view of the intraocular lens device shown in FIG. 1 in a resting position.

FIG. 3 is a side elevational view of the intraocular lens device shown in FIG. 1, when tension forces are applied to opposite ends of the haptic portions.

FIG. 4 is a top planar view of the intraocular lens device shown in FIGS. 1, 2 and 3.

FIG. 5 is a side elevational view of a second embodiment of the intraocular lens device according to the present invention shown in a resting position.

FIG. 6 is a side elevational view of the intraocular lens device shown in FIG. 5, when tension forces are applied to opposite ends of the haptic portions.

FIG. 7 is a top planar view of a third embodiment of an intraocular lens device according to the present invention.

FIG. 8 is a cross-sectional view of the intraocular lens device, as indicated in FIG. 7.

FIG. 9 is a cross-sectional view of the intraocular lens device, as indicated in FIG. 7.

FIG. 10 is a side elevational view of a fourth embodiment of an intraocular lens device according to the present invention, in a resting position.

FIG. 11 is a side elevational view of the intraocular lens device shown in FIG. 10, however, when tension force is applied to opposite ends of the haptic portions.

FIG. 12 is a top planar view of a fifth embodiment of the intraocular lens device according to the present invention.

FIG. 13 is a cross-section view of the intraocular lens device, as indicated in FIG. 12.

FIG. 14 is a top planar view of a sixth embodiment of the intraocular lens device according to the present invention.

FIG. 15 is a side elevational view of the intraocular lens device shown in FIG. 14.

FIG. 16 is a cross-sectional view of the intraocular lens device, as indicated in FIG. 14.

FIG. 17 is a top planar view of a seventh embodiment of the intraocular lens device according to the present invention.

FIG. 18 is a side elevational view of the intraocular lens device shown in FIG. 17.

FIG. 19 is a cross-sectional view of the intraocular lens device, as indicated in FIG. 17.

FIG. 20 is a partial broken away perspective view of the matrix lens insert of the intraocular lens device shown in FIG. 17.

FIG. 21 is a top planar view of an eight embodiment of the intraocular lens device according to the present invention.

FIG. 22 is a cross-sectional view of the intraocular lens device, as indicated in FIG. 21.

FIG. 23 is a partial broken away side elevational view of the leg portion of the intraocular lens device shown in FIG. 1.

FIG. 24 is a partial broken away side elevational view of the leg portion of the intraocular lens device shown in FIG. 5.

FIG. 24 is a partial broken away side elevational view of the leg portion of another intraocular lens device according to the present invention (not shown).

FIG. 26 is a partial broken away top planar elevational view of the leg portion of a further intraocular lens device according to the present invention (not shown).

FIG. 27 is a partial broken away side elevational view of the leg portion of another intraocular lens device according to the present invention (not shown).

### DETAILED DESCRIPTION OF PREFERRED EMBODIMENTS

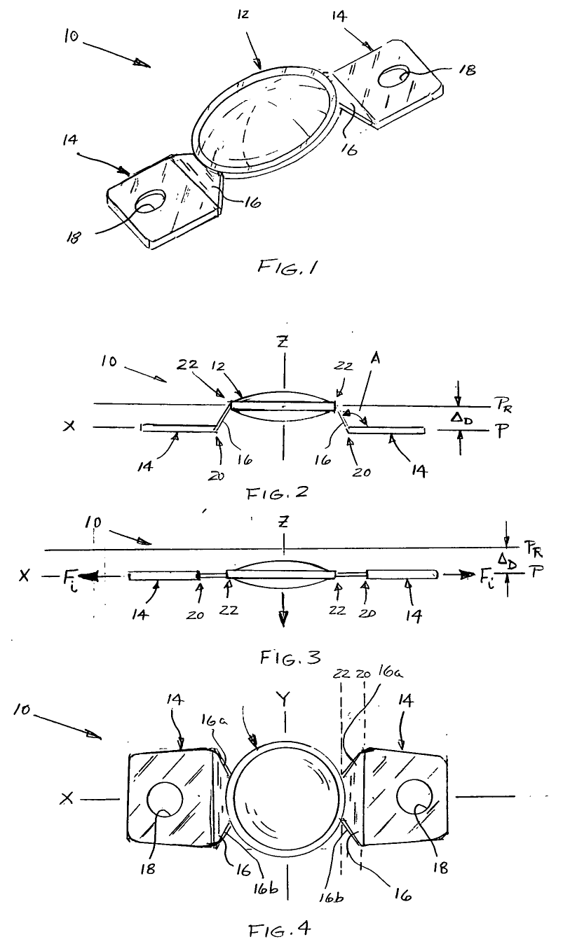

An intraocular lens device 10, in particular an accommodating intraocular lens device is shown in FIGS. 1 to 4.

The intraocular lens device 10 includes a lens portion 12 connected to plate haptic portions 14, 14 by leg portions 16, 16 located on opposite ends of the lens portion 12.

As shown in FIG. 2, the leg portions 16, 16 are bent relative to the lens portion 12 and the plate haptic portions 14, 14 when the intraocular lens device 10 is in a resting position. When tension forces Fᵢ are applied to opposite ends of the plate haptic portions 14, 14, the leg portions 16, 16 unbend and straighten out until aligned or substantially aligned with the direction of the tension forces Fᵢ. In this manner, the lens portion 12 traverses a distance Δ_{D} to provide accommodation of the intraocular lens device 10.

As shown in FIG. 4, the bent leg portions 16, 16 taper inwardly from the width of the plate haptic portions 14 to the connections with the len portion 12. The leg portions 16, 16 have inwardly tapering edges 16a and 16b. Preferably, the thickness of the leg portions 16, 16 is less than the plate haptic portions 14, 14 to facilitate unbending and straightening of the leg portions 16, 16 with the plate haptic portions 14, 14 when the tensions forces are applied.

The leg portions 16, 16 unbend relative to live hinge axis 20, 20 and live hinge axis 22, 22, as shown in FIG. 4.

The leg portions 16, 16 are set at an angle A relative to the plate haptic portions 14, 14, as shown in FIG. 2. The angle A is preferably 45° to a 135°. The angle A can be adjusted to adjust or control the distance Δ_{D} desired or prescribed for a particular patient or application.

A second embodiment of the intraocular lens device 110 according to the present invention is shown in FIG. 5.

The intraolcular lens device 110 includes a lens portion 112 connected to a pair of plate haptic portions 114, 114 by leg portions 116, 116. In this embodiment, the angle A between the plate haptic portion 114 and the leg portion 116 is approximately ninety degrees (90°). This arrangement will provide the maximum throw distance Δ_{D} during operation thereof. The intraocular lens device 110 is shown in the resting position, and when a tension force is applied to the opposite ends of the plate haptic portions 114, the leg portions 116 unbend relative to the lens portion 112 and the plate haptic portions 114, and straighten out to some extent or to a full extent, as shown in FIG. 6, depending on the amount of the tension forces and the amount of accommodation required by the eye.

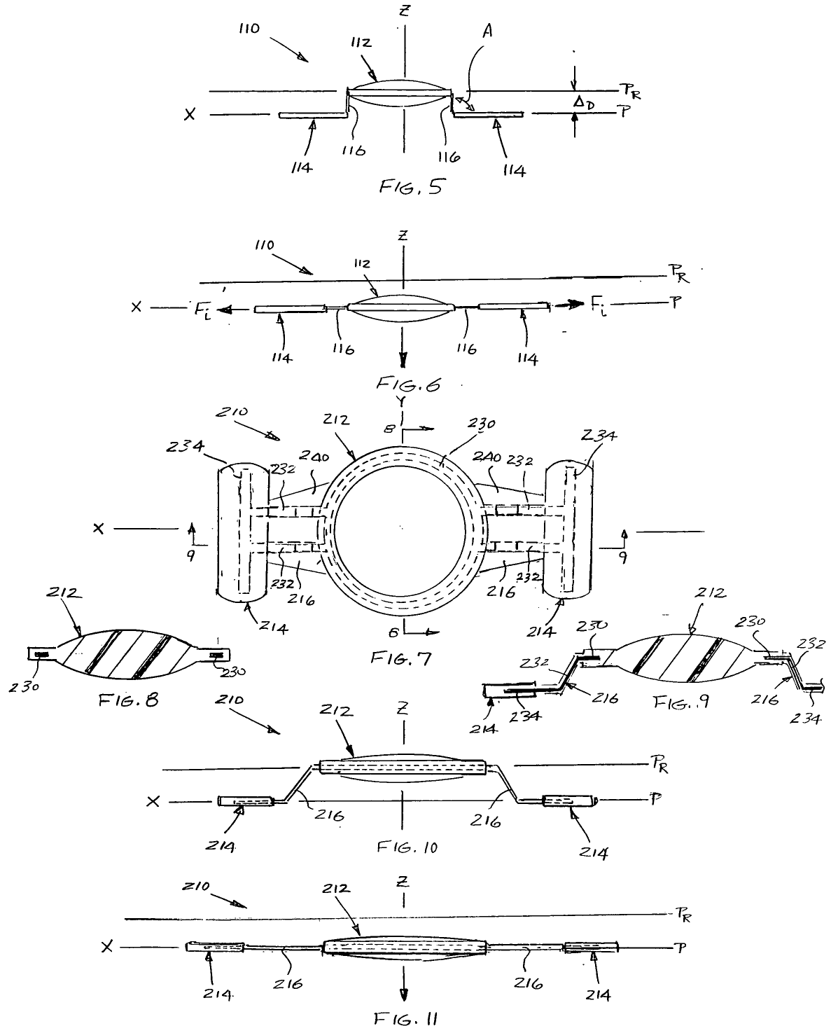

A third embodiment of the intraocular lens device 210 according to the present invention is shown in FIGS. 7 to 9.

The intraocular lens device 210 includes a lens portion 212 connected to a pair of plate haptic portions 214, 214 by leg portions 216, 216. The lens portion 212 is provided with a ring-shaped lens insert 230, as shown in FIGS. 7 and 8. The ring-shaped lens insert 230 can be made from a variety of materials the same as or different than the lens portion 212. For example, the lens insert 30 can be made of polypropylene, polyamide, polyimide, polyester, polycarbonate, polymethyl methacrylate, polysulfone, acrylic polymer or other suitable known material. The lens insert 230 is encapsulated within the resin material of the lens portion 212. In some embodiments, the ring-shaped lens insert 230 is unstressed during manufacturing of the intraocular lens device 210, or alternatively, the ring-shaped lens insert 230 is pre-stressed (e.g. compressive force applied to ring, tension force applied to ring, torsion force applied to ring, uniform tension force applied around ring, uniform compression force applied around ring, uniform torsional force applied around ring, local tension force applied to portion of ring, local compression force applied to ring, local torional force applied to ring, or combination thereof).

The leg portions 216, 216 are provided with leg inserts 232 configured to reinforce the leg portions 216, 216 along the X axis (i.e. unidirectional). The leg inserts 232 can be unstressed during manufacture of the intraocular lens device 210, or can be placed under tension, compression and/or tortional stresses along the lengths thereof, or at portions, or at points along the length thereof. The plate haptic portions 214, 214 are also provided with haptic inserts 234, 234, which reinforce the plate haptic portion 214 in the Y axis direction (i.e. unidirectional). The haptic inserts 234 can be unstressed during manufacture of the intraocular lens device 210, or alternatively, can be pre-stressed by tension, compression and/or tortional stresses prior to or during manufacturing of the intraocular lens device 210. Again, the pre-stressing forces can be applied uniformly, regionally, locally and/or as point forces.

The inserts 230, 232 and 234 can be separate pieces or components, or can be made as a single piece or unit. For example, when molding a deformable silicon intraocular lens, the inserts 230, 232 and 234 are made from a single sheet of polyamide or polyester and cut out (e.g. mechanically, waterjet) as a one-piece insert. The insert is then placed in the mold cavity, pre-stressed for example by pulling, pushing, twisting ends or edges of the one-piece insert, and then filling the mold cavity with a silicone polymer resin which encapsulates the one-piece insert during the molding process. Upon heating the mold to polymerize and cross link the silicone polymer resin, a one-piece pre-stressed intraocular lens device 210 is formed.

As an optional feature, a very thin section of resin is molded around the leg inserts 232 of the leg portions 216, 216 to provide webbing 240 to encapsulate the leg inserts 232 to protect the leg inserts 232 and/or to cover the leg inserts 232 so as not to damage tissues in the eye once implanted. For example, the thickness of the resin of the webbing 240 can be the same thickness as the plate haptic portions 214, but are preferably is of less thickness then the plate haptic portions 214, 214. In some embodiments, the thickness of the resin of the webbing 40 is less thickness of the leg inserts 232, and thus the legs inserts 32 with a resin layer thereon protrude outwardly to some extent from the surface or plane of the webbing 240 resulting in bumps on the surface thereof.

The inserts 230, 232 and 234, again can be made from a wide variety of materials, including but not limited to silicon polymer, acrylic polymer, polymethyl methacrylate (PMMA), polyamide, polyiimide, polyester, polycarbonate, polypropylene, fiber glass, Kevler, graphite, carbon fibers, ceramic, glass, metal, metal composite, polymer composite or other suitable known material. The material can be uniform throughout its dimensions, or can be fabricated to vary in thickness linearly, exponentially, continuously and/or discontinuously along the three-dimensional axes within insert. For example, the insert can be configured, designed, fabricated or otherwise made or tailored to vary in thickness, strength, shape, chemical composition, tensile strength, compressive strength, tortional strength, hardness, surface finish, surface texturing, or other lens or engineering parameters or variables typical of materials in one or more directions along the three-dimensional axes within the insert. The insert, for example, can have a variety of different transverse cross-sectional shapes such as a circle, triangle, square, rectangular, multi-sided (e.g. hexagonal), symmetrical, asymmetrical, tubing shaped, star-shaped, serrated edges, U-shaped, L1-shaped, etc. The insert can be a composite device such as a multi-layered or component implant, reinforced in one or more directions by inserts embedded within the insert, layers of varying degrees of polymerization along one or more dimensions thereof.

The surface of the inserts can be treated (e.g. radiated, chemically etched, heated, annealed, sanded, shot penned, glass beaded, roughened, machined) to facilitate adhesion and/or connection with the surrounding polymer resin material embedding the insert. Alternatively, the surface or portions of the surface of the insert can be configured or treated so that the insert does not adhere to the embedding polymer resin material so that the insert slides or slips within the material, even after polymerizing or cross-linking the embedding polymer resin material.

The inserts can be pre-stressed prior to placing in a mold cavity and/or in the mold cavity itself by applying tension forces, compressive forces and/or tortional force at one or more positions, areas or volumes within the insert material. Alternatively, or in addition, the insert can be pre-stressed by cooling, heating, steaming, radiating, curing, polymerizing, further polymerizing, or by other known methods or techniques prior to insertion in the mold, in the mold itself and/or after formation or manufacturing of the intraocular lens device. For example, the intraocular lens device is manufactured without pre-stressing the insert, however, the insert is treated with electromagnetic radiation (e.g. laser) prior to implantation in the eye. Of course, this intraocular lens device, in particular the insert of the intraocular lens device, can be further stressed or unstressed using electromagnetic radiation in vivo, after implantation within the eye.

A fourth embodiment of the intraocular lens device 310 according to the present invention is shown in FIGS. 12 and 13.

The intraocular lens device 310 includes a lens portion 312 connected to a pair of plate haptic portions 314, 314, by leg portions 316, 316. The lens portion 312 includes a circular-shaped lens insert 330a at or near the perimeter of the lens portion 312 and an inner circular-shaped lens insert 330b. The lens insert 330a has a rectangular or flat cross-sectional shape, as shown in FIG. 13, and the inner circular-shaped lens insert 330b has a circular cross-sectional shape, as shown in FIG. 13. The lens inserts 330a and 330b can be made from optically or substantially optically clear material (e.g. polypropylene), or can be made from partially or non-optically clear material, however, dimensioned so as to not optically interfere in a substantial or meaningful manner with light rays passing through the lens portion 312.

The lens inserts 230a and 230b, shown in FIGS. 12 and 13, may be significantly exaggerated in size versus the actual size of the implants for illustration purposes.

In the intraocular lens device 310, the leg portions 316, 316 are not provided with leg inserts to facilitate flexibility or bending thereof. The lens inserts 330a and 330b can be unstressed or pre-stressed in the manufactured intraocular lens device 310 prior to implantation in the eye. Once implanted in the eye, in vivo, the implants 330a and 330b can be treated with electromagnetic radiation uniformly, along portions thereof, and/or at point positions thereof to change the refractive characteristics of the lens portion (e.g. change aspheric lens properties of lens portions 312 and/or change the radius of curvature plus or minus at or near the locations of the implants).

The plate haptic portions 314 are provided with haptic implants 334, and configured in a manner to reinforce the plate haptic portions 314, 314 in two (2) dimensions along the plane of the plate haptic portions 314. Thus, the compressive strength between the opposite ends along the length of the plate haptic portions 314 are increased by the inserts 334 to facilitate accommodating tension forces applied along the length (i.e. along axis X of the intraocular lens device 310).

The plate haptic portions 314 are provided with through holes 318 to facilitate anchoring the plate haptic portions within the eye, in particular when implanted in the capsular bag of the posterior chamber of the eye. Alternatively, or in addition, the intraocular lens device 310 can be an implantable contact type intraocular lens such as an implantable contact lens (icl) or phakic refractive lens (prl).

A fifth embodiment of the intraocular lens device 410 according to the present invention is shown in FIGS. 14 to 16.

The intraocular lens device 410 is configured as a loop haptic type of intraocular lens device. The intraocualr lens device 410 includes a lens portion 412 connected to a pair of loop haptic portions 414, 414 by a pair of leg portions 416, 416.

The lens portion 412 is provided with a lens insert 430, which can be made by multiple pieces or as a single piece. The lens insert 430 includes circular-shaped lens insert portions 430a and 430b and 430c, and straight radial oriented lens insert portion 431a,b,c,d,e,f,h. As shown in FIG. 14, the straight radial oriented lens insert portions 431c and 431g are shown as tapering from a wider thickness at the center of the lens portion 412 to a thinner thickness towards the outer perimeter of the lens portion 412. This arrangement is to allow more thickening of the center portion of the lens portion when electromagnetic radiation, such as laser light, is applied to the thicker portions of the lens inserts 431c and 431g.

The loop haptic portions 414, 414 include a polymer resin layer 414a provided with haptic inserts 434 configured to reinforce and stiffen the loop haptic portions 414, 414. The leg portions 416, 416 are extensions of the haptic inserts 434, 434, and extend to the lens portion 412 where they are anchored by anchoring portions 438 in the perimeter of the lens portion 412.

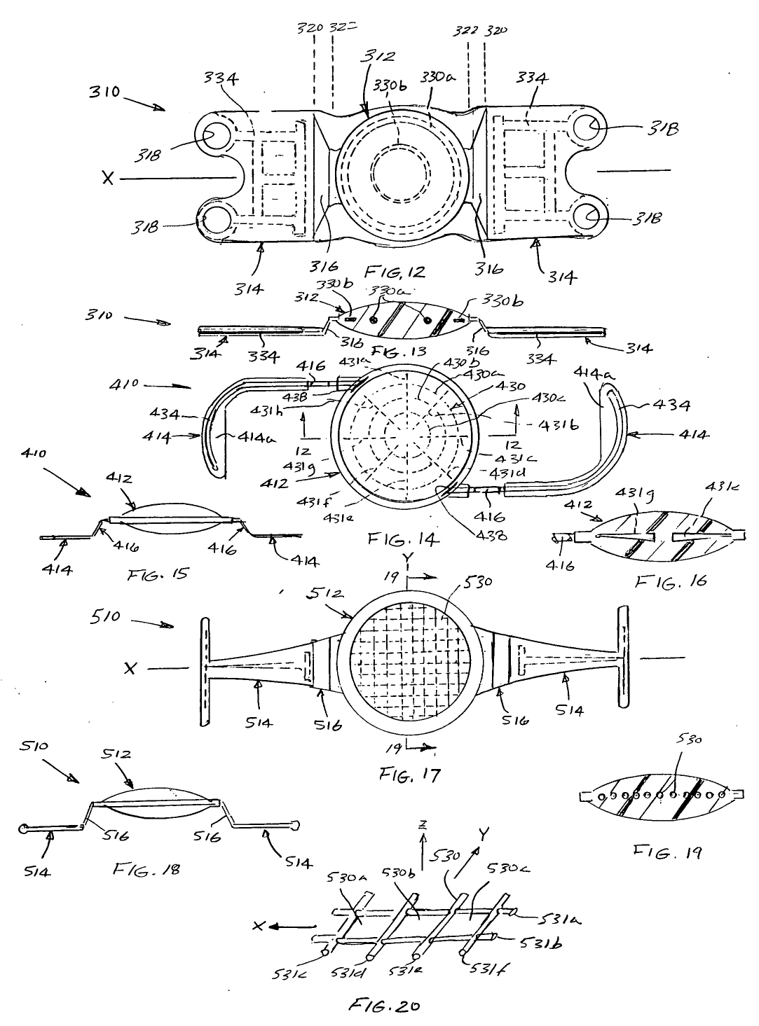

A sixth embodiment of the intraocular lens device 510 according to the present invention is shown in FIGS. 17 to 20.

The intraocular lens device 510 includes a lens portion 512 connected to a pair of haptic portion 514, 514 by a pair of leg portions 516, 516. The lens portion 512 is provided with an lens insert 530 configured as a matrix, screen or mesh. The matrix lens insert 530 can be made of separate pieces or as a single piece. The matrix lens insert 530 defines a matrix of rectangular, preferably square, cells (e.g. cells 530a and 530b, FIG. 20). However, the matrix can be configured to provide other shapes such as triangles, circles, multi-sided shapes, or can be made as a progressive matrix varying in the size of the openings in one or two or three dimensions. The matrix lens insert 530 is configures to accommodate precise treatment by electromagnetic radiation, in vivo, after implantation of the intraocular lens device 510 within the eye. The matrix arrangement facilitates locating specific points within the lens portion 512, and a particular portion or cell of matrix can be treated with the electromagnetic radiation to induce stress or unstress a particular point or region of the lens portion 512. For example, a portion of the matrix lens implant is hit with laser light to condense a particular cell or groups of cells of the matrix implant and/or the polymer resin material within a cell is treated with laser light to cause compression and expansion of the dimensions of a particular cell or groups of cells. In this manner, the thickness, curvature, stiffness, refractive light properties of the lens portion can be modified or varied in vivo, by treatment with electromagnetic radiation.

The matrix lens insert 530 can be a woven mesh of threads, wires, strands of material, or can be a single unwoven matrix (e.g. made by injection molding). Again, the matrix lens implant 530 is preferably made of optically clear material, or can be made from non-optically clear material, however dimensioned or sized so as to not significantly interfere with the light refractive properties of the lens portion 512.

As shown in FIG. 20, the matrix insert defines a plurality of cells 530a, 530b and 530c between horizontal strands 531a and 531b and vertical strands 531d-h. The matrix implant 530 is shown as being a non-woven mesh or screen configured in a single plane or configured to be shaped spherically (i.e. curved along the X axis and Y axis in the Z direction).

A seventh embodiment of the intraocular lens device 610 according to the present invention is shown in FIGS. 21 and 22.

The intraocular lens device 610 includes a lens portion 612 connected to a pair of plate haptic portions 614, 614 by a pair of leg portions 616, 616. The lens portion 612 includes a separate optical lens portion 612a connected to a separate outer lens ring 612b by a plurality of spoke members 640. The spoke members 640 can be extensions of lens inserts into the optical lens portion 612a and the outer lens ring portion 612b. The orientation, location, size, shape, configuration, refractive power, aspheric power and other parameters and characteristics of the optical portion can be changed or addressed by use of electromagnetic radiation, in vivo, after implantation of the intraocular lens device 610 into the eye. Specifically, the electromagnetic radiation can be focused or directed to specific spoke members 640 to the same or varying extent to provide tension along the spoke members or compression stresses in the spoke members.

A preferred embodiment of the intraocular lens device according to the present invention is an accommodating intraocular device. Specifically, the intraocular lens device is configured so that the lens portion thereof moves to some extent during operation of the eye to simulate the movement of the natural crystalline lens of the eye along the focal axis thereof prior to cataract removal. The accommodation function of the eye provides for a dioper change of the lens to facilitate a person's ability to read or look at object closely. The accommodating type intraocular lens device according to the present invention is to be used to restore accommodation for persons after a clear natural lens removal or a cataract lens removal, wherein the accommodating intraocular lens device is implanted into the capsular bag of the posterior chamber of the eye.

The intraocular lens device according to the present invention includes a lens portion connected to a hapic portion or pair haptic portions by one or more leg portions. The leg portions are preferably bent leg portions having a “bent” configuration when in a resting position. For example, the configuration of the intraocular lens device is shown in FIGS. 2, 5, 10, 13, 15, 18 and 22 show various embodiments of the intraocular lens devices in resting positions (i.e. no tension forces being applied to the ends of the haptic portions) with the leg portions being in the “bent” configuration. The intraocular lens device according to the present invention is configured so that the bent leg portions unbend and straighten when tension forces are applied to the ends of the haptic portion or portions causing the lens portion to move towards a plane containing the haptic portions. When the tension force is released, the leg portions are relaxed and go from a more straightened position to the original bent position.

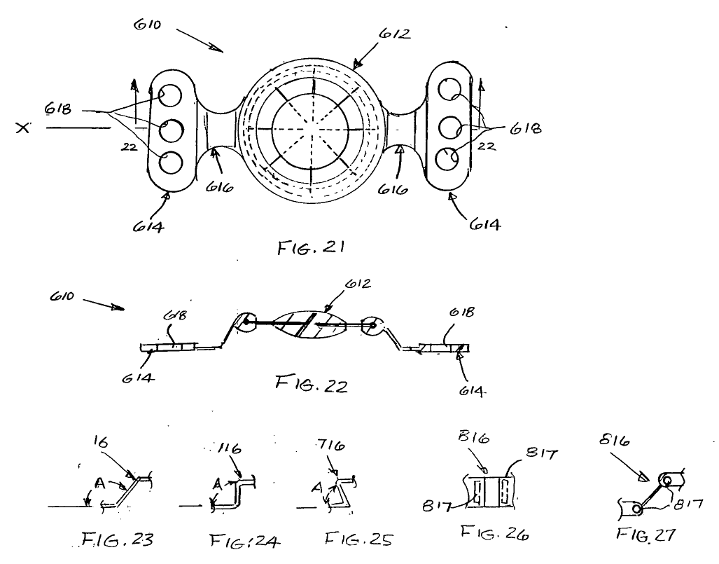

Various bent leg portions are shown in FIGS. 23 to 27.

The bent leg portions vary in angle relative to the lens portions and haptic portions of the intraocular lens device. The perpendicular embodiment shown in FIG. 24 provides maximum throw or movement of the lens portion relative to the haptic portion or portions. The angle A preferably ranges from 45 degrees to 135 degrees.

As shown in FIGS. 26 and 27, the leg portion 816 can be provided with rod-shaped implants 817, 817, configured to slid or slip within the thickness of the leg portions to enhance or facilitate bending along the bending axes thereof. The rod-shaped implants 817, 817 can be separate parts and operate independently, or can be linked or connected through the length of the leg portion 816 to reinforce the leg portion and still facilitate bending.

The intraocular lens according to the present invention can be implanted in the resting postion (e.g. FIGS. 2, 5, 10, 13, 15, 18 and 22) and heal in place within the eye in the resting position. Alterternatively, the intraocular lens are sprung or acutated (e.g. by sutures, removal connected stiffeners, manipulated in vivo, irradiated) when implanted in the eye and during the healing process, and then released only after significantly affixation of the haptic portions to the eye tissue (e.g. between the outer edges of the walls of the capsular bag).

I claim:

1. An accommodating intraocular lens device, comprising:
a lens portion located in a first plane;
at least one haptic portion located in a second plane located a predetermined distance from said first plane; and
at least one bent leg portion connecting said lens portion to said haptic portion, said bent leg portion configured to move said lens portion located in said first plane towards said at least one haptic portion located in said second plane when tension force are applied at opposite ends of said at least one haptic portion.

2. A lens device according to claim 1, wherein said lens portion is provided with at least one lens insert.

3. A lens device according to claim 1, wherein said at least one haptic portion is provided with at least one haptic insert.

4. A lens device according to claim 1, wherein said at least one leg portion is provided with at least one leg insert.

5. A lens device according to claim 2, wherein said at least one haptic portion is provided with at least one haptic insert.

6. A lens device according to claim 5, wherein said at least one leg portion is provided with at least one leg insert.

7. A lens device according to claim 2, wherein said lens insert is pre-stressed during making of said lens device.

8. A lens device according to claim 3, wherein said haptic insert is pre-stressed during making of said lens device.

9. A lens device according to claim 4, wherein said leg insert is pre-stressed during making of said lens device.

10. A lens device according to claim 1, wherein said leg portion is oriented at approximately 135° relative to said at least one plate haptic portion.

11. A lens device according to claim 1, wherein said leg portion is oriented at approximately 90° relative to said at least one plate haptic portion.

12. A lens device according to claim 1, wherein said first plane is oriented normal to said second plane.

13. A lens device according to claim 2, wherein said lens insert is a least one ring-shaped insert.

14. A lens device according to claim 13, wherein said lens insert is a plurality of concentric ring-shaped inserts.

15. A lens device according to claim 2, wherein said lens insert is at least one straight radially oriented lens insert.

16. A lens device according to claim 2, wherein said lens insert is a plurality of straight radially oriented lens inserts.

17. A lens device according to claim 13, wherein said lens insert includes at least one straight radially oriented lens insert.

18. A lens device according to claim 2, wherein said lens insert is a matrix lens insert.

19. A lens device according to claim 18, wherein said matrix lens insert includes rectangular cells.

20. A lens device according to claim 18, wherein said lens insert is a non-woven mesh.
